(19) United States
(12) Patent Application Publication
(10) Pub. No.: US 2025/0257304 A1
(43) Pub. Date: Aug. 14, 2025

(54) CELL DETACHMENT APPARATUS AND CELL DETACHMENT METHOD

(71) Applicant: CANON KABUSHIKI KAISHA, Tokyo (JP)

(72) Inventors: TATSUO FURUTA, Tokyo (JP); TAKANORI MATSUDA, Tokyo (JP); TAKAAKI FURUI, Tokyo (JP); RYOICHI YOSHIDA, Kanagawa (JP)

(21) Appl. No.: 19/194,574

(22) Filed: Apr. 30, 2025

Related U.S. Application Data
(63) Continuation of application No. PCT/JP2023/038838, filed on Oct. 27, 2023.

(30) Foreign Application Priority Data
Nov. 1, 2022 (JP) 2022-175809

(51) Int. Cl. C12M 1/26; C12M 3/00

(52) U.S. Cl. CPC C12M 33/08; C12M 23/48

(57) ABSTRACT

A cell detachment apparatus that detaches cells placed on a culture surface of a culture container for cells by applying vibration to the cells includes a vibrator and a vibrating plate, wherein the vibrator, the vibrating plate, an acoustic transmission medium, and the culture container are stacked in this order, wherein a bending vibration is excited on the culture container when a vibration of the vibrating plate is transmitted to the culture container via the acoustic transmission medium, and wherein the acoustic transmission medium and the culture container come into contact with each other at a region including an antinode of the bending vibration and do not come into contact at a node of the bending vibration.

### CROSS-REFERENCE TO RELATED APPLICATIONS

This application is a Continuation of International Patent Application No. PCT/JP2023/038838, filed Oct. 27, 2023, which claims the benefit of Japanese Patent Application No. 2022-175809, filed Nov. 1, 2022, both of which are hereby incorporated by reference herein in their entirety.

### BACKGROUND OF THE INVENTION

### Field of the Invention

The present invention relates to a cell detachment apparatus and a cell detachment method.

### Background Art

In the fields of cell pharmaceutical and regenerative medicine, a large number of cells needs to be cultured. In particular, it is demanded to effectively and stably supply adhesive cells occupying a large part of biological tissues.

In culturing adhesive cells, for example, cells are cultured on a culture container such as a polystyrene dish and detached from the culture container, and target cells are acquired through the cell collection and cleaning processes. To further increase the number of cells, some of the acquired cells are moved to a new culture container. This operation is called a successive operation. In this series of processes, the detachment process for detaching cells from the culture container is generally conducted by generating a flow in the liquid of the culture container by using a pipette to detach cells. However, differences in the pipette angle and generated flow between operators in the detachment process are obstacles for efficient and stable supply of cells.

Patent Document 1 discloses a cell collection apparatus for supplying a detachment solution for reducing the cell adhesiveness of a cultured cell group to a culture container storing a cultured cell group, and vibrating the culture container with a vibration apparatus to detach the cultured cell group from the inner wall of the culture container.

### CITATION LIST

### Patent Literature

PTL 1: Japanese Patent Laid-Open No. 2008-79554

Examples of usable culture containers include a dish, a flask, and a well plate. However, the bottom surface of a commercially available culture container for cell culturing may include a projecting portion or may not be flat (or actually bent). Consequently, when providing an acoustic transmission medium for transmitting a vibration between the bottom surface of the culture container and the vibration apparatus, air may enter between the bottom surface of the culture container and the acoustic transmission medium. Air remaining therebetween causes the vibration of the vibration apparatus to reflect on the interface between the acoustic transmission medium and air, or attenuate in air. As a result, the vibration of the vibration apparatus is less transmitted to the culture container, resulting in a reduced cell detachment efficiency. The air between the bottom surface of the culture container and the acoustic transmission medium cannot be removed unless deaeration processing is performed, which may possibly decrease the survival rate of the cells on the culture container. Therefore, to efficiently detach cells to obtain cells at a high cell detachment efficiency, it is necessary to prevent air from entering the gap between the acoustic transmission medium and the culture container.

### SUMMARY OF THE INVENTION

The present invention is directed to providing a cell detachment apparatus for detaching cells with a high cell detachment efficiency.

According to an aspect of the present invention, a cell detachment apparatus that detaches cells placed on a culture surface of a culture container for cells by applying vibration to the cells includes a vibrator and a vibrating plate, wherein the vibrator, the vibrating plate, an acoustic transmission medium, and the culture container are stacked in this order, wherein a bending vibration is excited on the culture container when a vibration of the vibrating plate is transmitted to the culture container via the acoustic transmission medium, and wherein the acoustic transmission medium and the culture container come into contact with each other at a region including an antinode of the bending vibration and do not come into contact at a node of the bending vibration.

According to another aspect of the present invention, a cell detachment method for applying a vibration to cells disposed on a culture surface of a cell culture container to detach the cells includes applying a vibration to the cells by using a cell detachment apparatus having a vibrator and a vibrating plate, an acoustic transmission medium, and the culture container in a state where the vibrator, the vibrating plate, the acoustic transmission medium, and the culture container are stacked in this order, and applying a vibration to the cells in a state where a bending vibration is excited on the culture container when a vibration of the vibrating plate is transmitted to the culture container via the acoustic transmission medium, and the acoustic transmission medium and the culture container come into contact with each other at a region including an antinode of the bending vibration but do not come into contact at a node of the bending vibration.

Further features of the present invention will become apparent from the following description of exemplary embodiments with reference to the attached drawings.

### BRIEF DESCRIPTION OF THE DRAWINGS

FIG. 1A is a perspective view illustrating a configuration of a cell detachment apparatus according to an exemplary embodiment of the present invention.

FIG. 1B is an exploded perspective view illustrating the configuration of the cell detachment apparatus according to an exemplary embodiment of the present invention.

FIG. 1C is a cross-sectional view illustrating a configuration of the cell detachment apparatus according to an exemplary embodiment of the present invention.

FIG. 2A is a schematic view illustrating a ring type vibrator used for the cell detachment apparatus according to an exemplary embodiment of the present invention.

FIG. 2B is a schematic view illustrating the ring type vibrator used for the cell detachment apparatus according to an exemplary embodiment of the present invention.

FIG. 3A is a bottom view illustrating a deformation state of a culture container by a bending vibration, used for the cell detachment apparatus according to an exemplary embodiment of the present invention.

FIG. 3B is a cross-sectional view illustrating the deformation state of the culture container by the bending vibration, used for the cell detachment apparatus according to an exemplary embodiment of the present invention.

FIG. 4 is a cross-sectional view illustrating a shape of the culture container used for the cell detachment apparatus according to an exemplary embodiment of the present invention.

FIG. 5A is a bottom view illustrating a deformation state of the culture container by a bending vibration, used for the cell detachment apparatus according to an exemplary embodiment of the present invention.

FIG. 5B is a cross-sectional view illustrating the deformation state of the culture container by the bending vibration, used for the cell detachment apparatus according to an exemplary embodiment of the present invention.

FIG. 6 is a cross-sectional view illustrating a configuration of the cell detachment apparatus according to an exemplary embodiment of the present invention.

FIG. 7A is a cross-sectional view illustrating a configuration of the cell detachment apparatus according to an exemplary embodiment of the present invention.

FIG. 7B is an exploded perspective view illustrating the configuration of the cell detachment apparatus according to an exemplary embodiment of the present invention.

FIG. 8A is a bottom view illustrating a deformation state of the culture container by a bending vibration, used for the cell detachment apparatus according to an exemplary embodiment of the present invention.

FIG. 8B is a cross-sectional view illustrating the deformation state of the culture container by the bending vibration, used for the cell detachment apparatus according to an exemplary embodiment of the present invention.

FIG. 9 is a cross-sectional view illustrating a configuration of the cell detachment apparatus according to an exemplary embodiment of the present invention.

### DESCRIPTION OF THE EMBODIMENTS

The present invention will be described in detail below with reference to preferred exemplary embodiments.

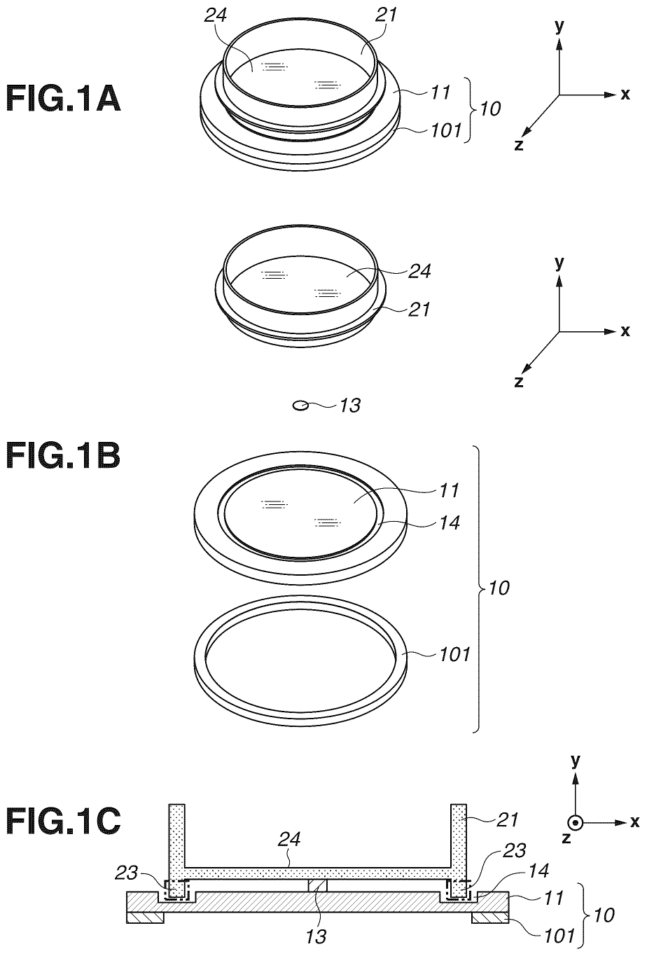

FIGS. 1A, 1B, and 1C are schematic views illustrating a cell detachment apparatus according to an exemplary embodiment of the present invention. FIG. 1A is a schematic perspective view illustrating a culture container 21 and a cell detachment apparatus 10 viewed from a diagonal direction. FIG. 1B is an exploded perspective view of FIG. 1A, and illustrates the culture container 21, an acoustic transmission medium 13, a vibrating plate 11, and a vibrator 101 separated in the center axis direction of the ring type vibrator 101. FIG. 1C is a schematic lateral view illustrating a center cross-section of the cell detachment apparatus 10 according to an exemplary embodiment of the present invention.

As illustrated in FIGS. 1A, 1B, and 1C, the cell detachment apparatus 10 according to an exemplary embodiment of the present invention includes the ring type vibrator 101 and the discoid vibrating plate 11. With the cell detachment apparatus of an exemplary embodiment of the present invention, the vibrator 101 is driven in a state where the vibrator 101, the vibrating plate 11, the acoustic transmission medium 13, and the culture container 21 are stacked in this order. When the vibration of the vibrator 101 is transmitted to the vibrating plate 11, and the vibration of the vibrating plate 11 is further transmitted to the culture container 21 via the acoustic transmission medium 13, a bending vibration is excited on the culture container 21. Thus, the cell detachment apparatus 10 applies a vibration to the cells disposed on the culture surface of the culture container 21 to detach the cells. Another member may be provided between the vibrator 101 and the vibrating plate 11, between the vibrating plate 11 and the acoustic transmission medium 13, or between the acoustic transmission medium 13 and the culture container 21.

The vibration to be applied may be a vibration in the ultrasonic range. The vibration in the ultrasonic range is a vibration having a frequency of 20 kHz or higher. The acoustic transmission medium 13 may be pre-installed on the cell detachment apparatus 10, or the acoustic transmission medium 13 may not be pre-installed on the cell detachment apparatus 10 but may be installed on the cell detachment apparatus 10 by the user. The vibrating plate 11 may be provided with a marker (such as a label, print, and marking) indicating the position where the acoustic transmission medium 13 is to be disposed on the vibrating plate 11. The marker on the vibrating plate 11 allows the user to easily place the acoustic transmission medium 13 at a suitable position.

The cell detachment apparatus 10 may further include a control unit for driving the vibrator 101 to apply a vibration to the cells. The acoustic transmission medium 13 can also be referred to as an acoustic transmission material, acoustic matching material, or transmission medium.

The term “detaching cells” refers to weakening the adhesion of cells to the culture container 21 to allow the cells to be collected. At this timing, the detached cells may exist as individual cells, or a plurality of cells may be adhered to each other. The detached cells may form a sheet (described below).

### (Ring and Discoid Shapes)

According to the present specification, the ring shape refers to a discoid shape having a predetermined thickness and a circular through-hole, and the outer circumference and the through-hole are concentric. The discoid shape refers to a circular shape having a predetermined thickness. This circular shape and the outer circumferential shape of the through-hole are ideally a perfect circle but may be an oval or ellipse as long as they can be schematically considered as a circle. If a circle is not a perfect circle, the radius and diameter of the circle are defined as the length of the figure in the long axis direction, i.e., the major axis. If a circle is not a perfect circle, the radius and diameter of the circle may be determined on the assumption of a perfect circle having the same area.

A ring or discoid shape having a missing or projecting portion are also included in the ring or discoid shape, respectively. Therefore, even if a ring or discoid shape is slightly deformed by variation in the production, the shape is included in the ring or discoid shape as long as the shape can be substantially considered as a ring or discoid shape. With a shape having a missing or projecting portion, the inner diameter, outer diameter, and diameter for a ring shape and the radius and diameter for a discoid shape are determined on the assumption of a perfect circle with the missing or abnormal portion corrected.

The cell detachment apparatus according to an exemplary embodiment of the present invention will be described below for a case where a ring type vibrator and a discoid vibrating plate are provided. However, the shapes of these members are not limited thereto.

### (Vibrator)

The type of the vibrator 101 may be either the piezoelectric or electromagnetic type, and is not particularly limited as long as a vibration of a certain frequency can be generated. If the vibrator 101 is of the piezoelectric type, the vibrator 101 may be selected from the unimorph type, bimorph type, and bolt-clamped Langevin type. The material and structure of the vibrator 101 may be selected according to the desired frequency, force, and vibration amplitude. Referring to FIGS. 1A, 1B, and 1C, the vibrator 101 having a ring shape and the vibrating plate 11 having a discoid shape are of the unimorph type of the piezoelectric type.

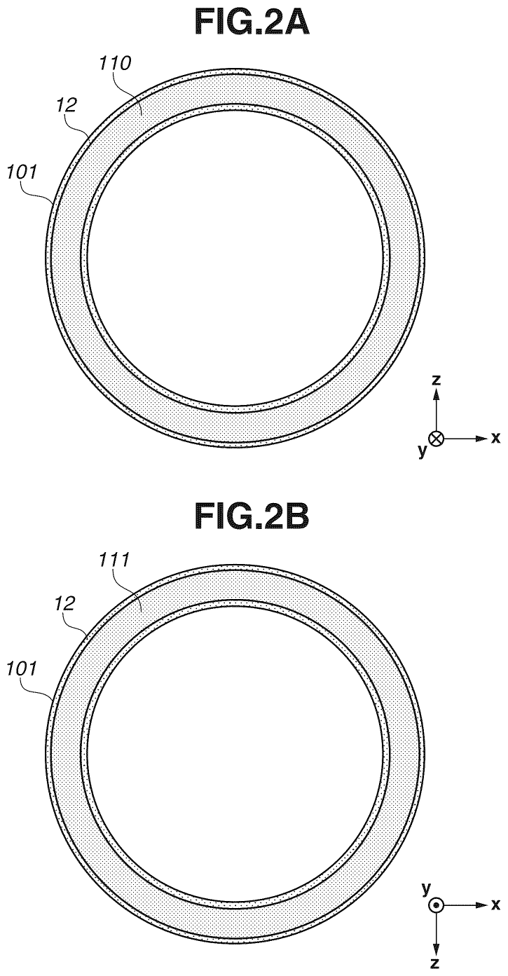

FIGS. 2A and 2B are schematic views illustrating the vibrator 101 used for the cell detachment apparatus 10 according to an exemplary embodiment of the present invention. FIG. 2A illustrates the ring type vibrator 101 viewed from the direction opposite to the side of the vibrating plate 11 (direction from the origin to the y direction in FIG. 1C). FIG. 2B illustrates the vibrator 101 viewed from the side of the vibrating plate 11 (direction from the origin to the negative direction of the y axis in FIG. 1C).

The vibrator 101 includes a ring shape piezoelectric material 12, a common electrode 111 disposed on a first surface of the piezoelectric material 12 (the side of the vibrating plate 11, i.e., the y direction in FIG. 1C), and a driving electrode 110 disposed on a second surface opposite to the side of the vibrating plate 11 (negative direction of the y axis in FIG. 1C). When an alternating voltage is generated between the driving electrode 110 and the common electrode 111, the piezoelectric material 12 expands and contracts. Accordingly, the surface of the vibrating plate 11 on the vibrator side expands and contracts in the in-plane direction, and the vibrating plate 11 bends in the out-place direction during vibration. Referring to FIGS. 1A, 1B, and 1C, the common electrode 111 and the driving electrode 110 are not illustrated.

### (Piezoelectric Material)

The form of the piezoelectric material 12 of the vibrator 101 according to an exemplary embodiment of the present invention is not limited and may be ceramics, powder, monocrystal, or slurry. Preferably, the piezoelectric material 12 is ceramics. According to the present specification, the term “ceramics” refers to a material having a metal oxide as the basic component and which is an aggregate (also referred to as a bulk body) of crystal grains sintered by heat processing, or what is called multicrystal. Ceramics also include a material processed after sintering. The composition of the piezoelectric material 12 is not particularly limited. Examples of materials usable for the vibrator 101 include the piezoelectric material 12 having at least one composition selected from a group including lead zirconate titanate, barium titanate, zirconic acid titanate, bismuth sodium titanate, potassium sodium niobate, barium sodium niobate titanate, and bismuth ferrate, or the piezoelectric material 12 having these compositions as the principal components.

Preferably, the piezoelectric material 12 according to an exemplary embodiment of the present invention has a mechanical quality factor of 1,000 or larger.

### (Mechanical Quality Factor)

The mechanical quality factor refers to a coefficient representing the elastic loss by vibration when the piezoelectric material 12 is evaluated as the vibrator 101. The magnitude of the mechanical quality factor is observed as the sharpness of the resonance curve in impedance measurement. In other words, the mechanical quality factor is a constant representing the sharpness of the resonance of the vibrator 101. The elastic loss causes generation of heat and this heat transmitted to cells may kill the cells or cause the development of an abnormal cell type. If the piezoelectric material 12 has a mechanical quality factor of 1,000 or larger, the elastic loss of the vibrator 101 using the piezoelectric material 12 decreases, thus reducing the heating value of the vibrator 101 during vibration.

### (Measurement of Piezoelectric Constant and Mechanical Quality Factor)

The mechanical quality factor of the piezoelectric material 12 can be obtained through calculation conforming to Japan Electronics and Information Technology Industries Association (JEITA) standard EM-4501 based on the result of measuring the resonant and antiresonant frequencies obtained by using a commercial impedance analyzer. Hereinafter, this method is referred to as a resonance-to-antiresonance method.

If the piezoelectric material 12 of the vibrator 101 has a mechanical quality factor of 1,000 or larger, a vibration loss is small and a heating value during vibration is small, and heat is less likely to be transmitted to the cells in the culture container 21.

### (Detection Electrode)

The vibrator 101 according to an exemplary embodiment of the present invention may include a detection electrode. The detection electrode has a function of monitoring the vibration state of the vibrating plate 11 by detecting the positive piezoelectric effect generated by the bending vibration of the vibrating plate 11 as voltage variations occurring in the piezoelectric material 12 sandwiched by the common electrode 111 and the detection electrode. The vibrator 101 having the detection electrode is able to feed the driving state back to driving conditions or detect a failure.

### (Characteristics, Materials, and Forming Methods of Electrodes)

The detection electrode, the common electrode 111, and the driving electrode 110 are formed of layer- or film-like conductors having a resistance value of less than 10 ohms, preferably less than 1 ohm. The resistance values of these electrodes can be measured by using, for example, a circuit tester (multimeter). The thickness of each electrode is about 5 nm to 20 μm. The materials of these electrodes are not particularly limited as long as the materials are ordinarily used for the piezoelectric material 12.

Examples of electrode materials include at least one metal selected from a group including Ti, Pt, Ta, Ir, Sr, In, Sn, Au, Al, Fe, Cr, Ni, Pd, Ag, and Cu, and their compounds. These electrodes may be formed of one of the above-described materials or two or more different materials stacked in layers. Of these, as the electrode used in an exemplary embodiment of the present invention, an Ag paste or Ag baked electrode and an Au/Ti sputtering electrode are preferable because of their small resistance values.

### (Shape of Vibrating Plate)

The shape of the vibrating plate 11 is not particularly limited as long as the vibration of the vibrator 101 can be transmitted to the culture container 21 via the acoustic transmission medium 13. For example, if the vibrator 101 is of the bolt-clamped Langevin type, the vibrating plate 11 may be formed of a cone or horn so that the desired vibration amplitude is generated. If the vibrator 101 is of the unimorph type, preferably, the vibrating plate 11 has a thin plate-like shape.

However, at the end of the surface opposite to the culture surface of the culture container 21, there may be provided a projecting portion 23 projecting in the direction approximately perpendicular to the surface on the opposite side. Preferably, the acoustic transmission medium 13 and the vibrating plate 11 are configured such that they do not come into contact with the projecting portion 23 when the culture container 21 is placed on the acoustic transmission medium 13.

The projecting portion 23 projects toward the acoustic transmission medium 13 like the portion enclosed in dot-dash lines in FIG. 1C. When the culture container 21 is stacked on the acoustic transmission medium 13, if the acoustic transmission medium 13 and the vibrating plate 11 come into contact with the projecting portion 23, it reduces the vibrations at the contact portions, possibly reducing the bending vibration to be excited on the culture container 21.

If a groove 14 is provided on the vibrating plate 11 so that the projecting portion 23 does not come into contact with the vibrating plate 11, as illustrated in FIG. 1B, which is preferable, because the bending vibration to be excited on the culture container 21 is not reduced. In other words, when the acoustic transmission medium 13 and the culture container 21 are stacked on the cell detachment apparatus 10 in this order as illustrated in FIG. 1C, desirably, the groove 14 is disposed in a region including the position on the vibrating plate 11 where the position of the projecting portion 23 is projected in the stacking direction (y direction in FIG. 1C).

The projecting portion 23 of the culture container 21 and the projecting portion 23 not coming into contact with the vibrating plate 11 refers to the state where the projecting portion 23 of the culture container 21 substantially does not come into direct contact with the vibrating surface of the vibrating plate 11. Preferably, the projecting portion 23 is floating. There may be provided a certain amount of a buffer material to the extent that the vibration of the vibrating plate 11 is not transmitted from the projecting portion 23 to the culture container 21. In other words, at least either one of the acoustic transmission medium 13 and the vibrating plate 11 comes into contact with the projecting portion 23 via the buffer material, and the acoustic transmission medium 13 and the vibrating plate 11 do not contact with the projecting portion 23 without the buffer material. Preferably, the buffer material is at least either one of felt and sponge.

Preferably, the surface of the vibrating plate 11 on which the vibrator 101 is disposed is preferably a flat surface since it provides favorable vibration transmission accompanying the expansion and contraction of the piezoelectric material 12. The method for disposing the vibrator 101 on the surface of the vibrating plate 11 is not limited. However, preferably, the vibrator 101 is closely bonded to the surface not to disturb the vibration transmission or closely bonded to the surface via a highly elastic adhesive layer (not illustrated). For example, an epoxy-based resin is preferably used as the adhesive layer.

### (Material of Vibrating Plate)

Preferably, the vibrating plate 11 is an elastic member since it is intended to generate an out-of-plane vibration together with the vibrator 101 and transmit the vibration to the culture container 21 via the acoustic transmission medium 13. From the viewpoint of the characteristics and processability of an elastic member, preferably, the vibrating plate 11 contains at least one material selected from a group including glass, metals, and quartz.

Examples of metals usable for the vibrating plate 11 include at least one material selected from a group including aluminum, brass, Fe—Ni 36% alloy, and stainless steel (SUS).

### (Acoustic Transmission Medium)

The method for disposing the acoustic transmission medium 13 on the surface of the vibrating plate 11 is not limited. However, preferably, the acoustic transmission medium 13 is brought into pressure contact with and closely bonded to the surface not to disturb the vibration transmission, or closely bonded to the surface via a highly elastic adhesive layer (not illustrated). In the case of close bonding with pressure contact, the adhesiveness is improved by smoothing the surface of the acoustic transmission medium 13.

### (Descriptions of Contact Position Between Acoustic Transmission Medium and Culture Container, and Bending Vibration)

When the vibration of the vibrating plate 11 is transmitted to the culture container 21 via the acoustic transmission medium 13, a bending vibration is excited on the culture container 21. The excited bending vibration may have a plurality of antinodes. At this time, the acoustic transmission medium 13 and the culture container 21 come into contact with each other at regions including at least a part of a plurality of antinodes, but not at nodes of the bending vibration. Examples of regions where the acoustic transmission medium 13 and the culture container 21 come into contact with each other may include a region including only an antinode, a region between an antinode and a node, and a region including an antinode but no node.

The bending vibration described in the present specification provides the natural vibration mode of the culture container 21, and a bottom surface 24 of the culture container 21, which is a culture surface, provides the natural vibration mode having an amplitude in the out-of-plane direction. The bending vibration according to an exemplary embodiment of the present invention provides higher order natural vibration mode having a plurality of antinodes. The natural vibration mode refers to a deformation state of the culture container 21 driven at the natural vibration frequency at which the vibration amplitude becomes maximal when the cell detachment apparatus is driven.

According to an exemplary embodiment of the present invention, the bending vibration refers to a standing wave with which the vibration amplitude of the bottom surface of the culture container 21 in the out-of-plane direction becomes maximal when the vibration having any desired frequency is transmitted to the culture container 21. With the bending vibration, a portion having a maximal amplitude is an antinode, and a portion having a minimal amplitude is a node.

The vibration transmitted to the culture container 21 is vibration that is generated by the vibration of the vibrator 101 being transmitted to the vibrating plate 11, and the vibration of the vibrating plate 11 being transmitted to the culture container 21 via the acoustic transmission medium 13. Therefore, the frequency of the vibration transmitted to the culture container 21 coincides with the frequency of the alternating voltage applied to the vibrator 101.

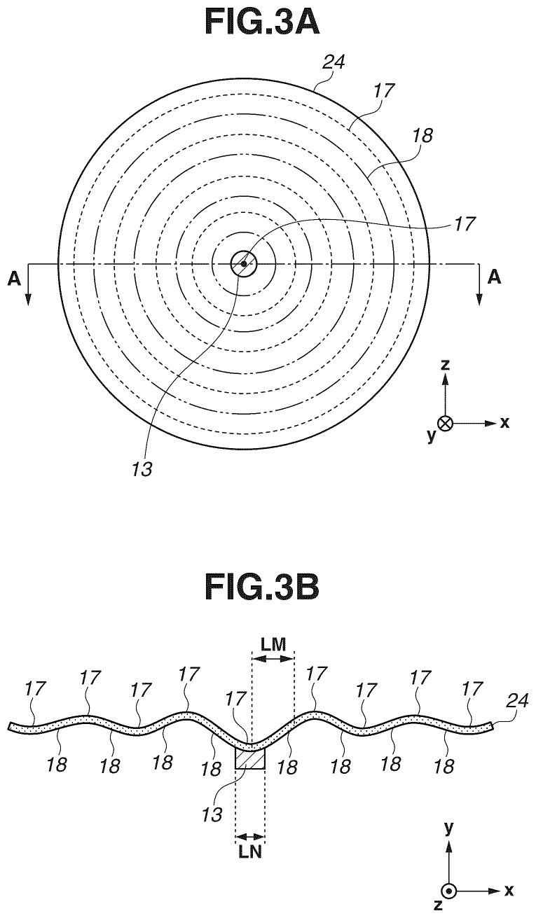

FIGS. 3A and 3B schematically illustrate the deformation state of the natural vibration mode in which the acoustic transmission medium 13 and the bottom surface 24 of the culture container 21 in FIGS. 1A, 1B, and 1C enter a bending vibration state. FIG. 3A is a plan view illustrating the culture container 21 viewed from below the culture container 21. FIG. 3B is a lateral view illustrating a center cross-section (taken along the dot-dash line A-A of FIG. 3A) of the bottom surface 24 of the culture container 21 in the deformation state. However, referring to FIG. 3B, the deformation amount is illustrated with the increased deformation rate. Referring to FIG. 3A, the black circle at the center and the broken lines denote antinodes 17, and the chain double-dashed lines denote nodes 18.

Referring to FIG. 3B, the acoustic transmission medium 13 is disposed at the position corresponding to the antinode 17 of the bending vibration on the bottom surface 24 of the culture container 21, and is not disposed at the nodes 18. In this case, the acoustic transmission medium 13 and the culture container 21 come into contact with each other at the antinode 17 of the bending vibration and does not at the nodes 18 of the bending vibration.

Thus, since the vibrations of the vibrator 101 and the vibrating plate 11 are transmitted to the antinodes 17 of the bending vibration on the culture container 21 via the acoustic transmission medium 13, the bending vibration of bottom surface 24 of the culture container 21 is strongly excited, resulting in the increased vibration amplitude of the bending vibration. If the acoustic transmission medium 13 is disposed at a node 18 of the bending vibration, the vibration of the vibrator 101 is transmitted to the node 18 of the bending vibration, and a force is applied to the bottom surface 24 to cause a vibration having a shape different from the natural vibration mode. Therefore, the excitation of the bending vibration is attenuated and the vibration amplitude decreases. As a result, detaching the cells in the culture container 21 becomes difficult.

Preferably, the acoustic transmission medium 13 is also disposed around the antinodes 17 of the bending vibration on the culture container 21 to the extent that the acoustic transmission medium 13 does not come into contact with the nodes 18 of the bending vibration. On the other hand, in the primary bending vibration, the vicinity of the edge of the culture container 21 corresponds to a node 18. Therefore, the vibration of the vibrator 101 cannot be efficiently transmitted to the culture container 21 unless the acoustic transmission medium 13 is disposed on almost the entire range of the bottom surface 24.

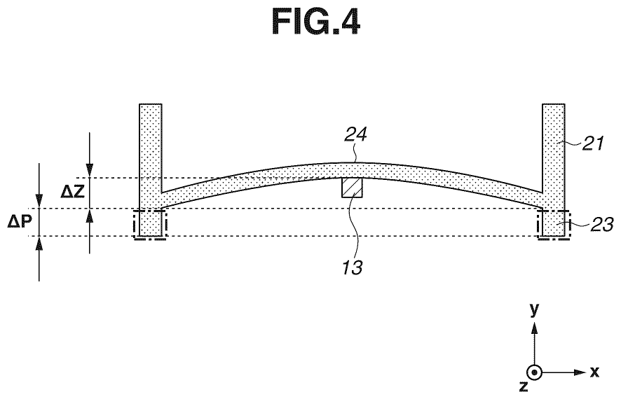

FIG. 4 illustrates a warpage on the bottom surface 24 of the culture container 21 in FIG. 1C, with the amount of warpage emphasized. An amount of warpage AZ depends on the size of the culture container 21. For example, the culture container 21 having a 51.6 mm outer diameter (Corning Incorporated) has a variation from 0.15 mm to 0.47 mm. If the acoustic transmission medium 13 is disposed on almost the entire range of the bottom surface 24 of the culture container 21 having such a warpage, air enters the center portion of the warpage and prevents the acoustic transmission medium 13 from coming into contact with the antinode 17 of the bending vibration.

Therefore, with the secondary or higher-order bending vibration, the distance between the antinode 17 and the node 18 is reduced, and the acoustic transmission medium 13 to be disposed is also substantially reduced in size. Accordingly, air is less likely to enter between the culture container 21 and the acoustic transmission medium 13, allowing the vibration of the vibrator 101 to be easily transmitted to the culture container 21.

In a case where the bending vibration having three or more antinodes 17, more preferably five or more antinodes 17, is excited on the culture container 21, desirably, the acoustic transmission medium 13 and the culture container 21 come into contact with each other at the antinodes 17 of the bending vibration but does not come into contact at the nodes 18 of the bending vibration. Accordingly, air is less likely to enter between the culture container 21 and the acoustic transmission medium 13, preferably allowing the vibration of the vibrator 101 to be easily transmitted to the culture container 21.

Preferably, LN1/LM is 1.0 or larger and 1.6 or less where LN1 denotes the diameter of the region where the acoustic transmission medium 13 and the culture container 21 come into contact with each other, and LM denotes the distance from the first antinode of the bending vibration to the first node of the bending vibration closest to the first antinode 17. If LN1/LM is 1.0 or larger, the region subjected to the vibration transmission is enlarged, allowing the vibration of the vibrator 101 to be efficiently transmitted to the culture container 21. If LN/LM is 1.6 or less, air is less likely to enter between the culture container 21 and the acoustic transmission medium 13.

Preferably, the bending vibration having a plurality of antinodes 17 excited on the culture container 21 by the vibrator 101 via the acoustic transmission medium 13 includes a plurality of different natural vibration modes.

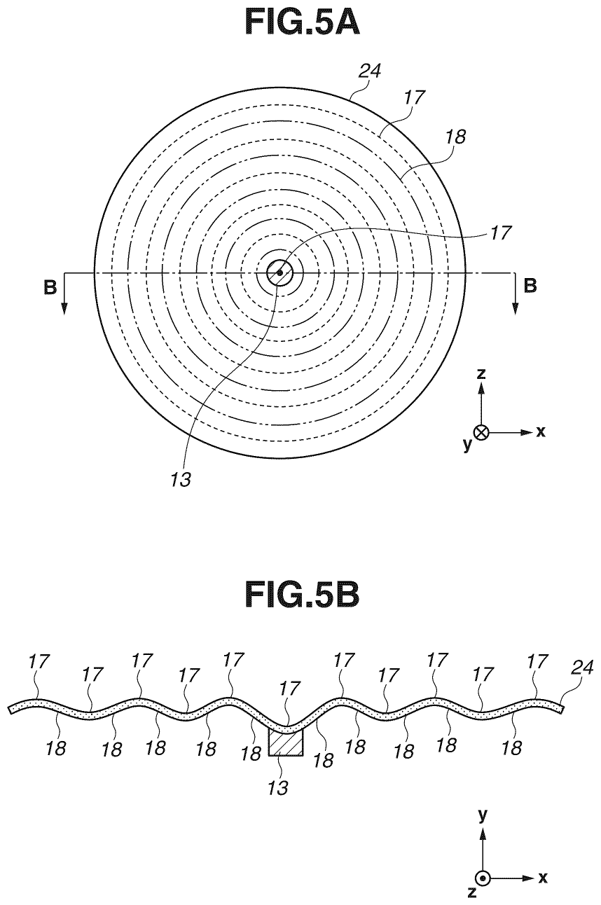

FIGS. 5A and 5B schematically illustrate a deformation state of the bending vibration having the natural vibration mode different from that in FIGS. 3A and 3B, by extracting the acoustic transmission medium 13 and the bottom surface 24 of the culture container 21 in FIGS. 1A, 1B, and 1C.

FIG. 5A is a plan view illustrating the culture container 21 viewed from below the culture container 21. FIG. 5B is a lateral view illustrating a center cross-section (taken along the dot-dash line B-B of FIG. 5A) of the bottom surface 24 of the culture container 21 in the deformation state. Referring to FIG. 5A, the black circle and the broken lines denote the antinodes 17, and the chain double-dashed lines denote the nodes 18.

Only with the natural vibration mode of the bending vibration in FIG. 3A, the cells are hardly detached because the vibration amplitude of a node 18 of the bending vibration excited on the culture container 21 is minimal. Therefore, when detaching the cells at the node 18, preferably, the bending vibration of a natural vibration mode different from the one in FIGS. 3A and 3B, as illustrated in FIGS. 5A and 5B, may be applied to the relevant node 18. If the bending vibration to be excited on the culture container 21 has a plurality of different natural vibration modes, the nodes 18 at almost different positions can be vibrated by each other, thus detaching the cells.

When exciting a plurality of different natural vibration modes in this way, the excitation of different natural vibration modes can be implemented by changing the drive frequency to the frequency at which each natural vibration mode is excited. The excitation of different natural vibration modes can also be implemented by sweeping the drive frequency within a range including the frequency at which each natural vibration mode is excited.

When exciting a plurality of different natural vibration modes, desirably, the acoustic transmission medium 13 is disposed at the position of the antinode 17 common to the bending vibrations of different natural vibration modes. If the acoustic transmission medium 13 is not disposed at the nodes 18 of the bending vibration, the acoustic transmission medium 13 and the culture container 21 come into contact with each other at the antinodes 17 of the bending vibration but does not come into contact at the nodes 18 of the bending vibration.

### (Shape of Acoustic Transmission Medium)

Although the shape of the acoustic transmission medium 13 is not particularly limited, the cuboid, ring, and discoid shapes are preferable from the viewpoint of easiness to process. The acoustic transmission medium 13 illustrated in FIGS. 1A, 1B, and 1C is discoid. In other words, the acoustic transmission medium 13 has a circular cross-section in a plane perpendicular to the direction in which the acoustic transmission medium 13 and the culture container 21 are stacked.

Preferably, the acoustic transmission medium 13 at the portion where the acoustic transmission medium 13 comes into contact with the culture container 21 has a thickness of 0.1 mm or more and 3 mm or less. The acoustic transmission medium 13 having a thickness of 3 mm or more attenuates the vibration in the acoustic transmission medium 13, and the vibration of the vibrator 101 is not transmitted to the culture container 21. The acoustic transmission medium 13 having a thickness of 0.1 mm or less is difficult to manufacture. Preferably, the acoustic transmission medium 13 has a thickness of 0.1 mm or more.

Preferably, the surface of the acoustic transmission medium 13 in contact with the culture container 21 includes a convex curved surface. If the acoustic transmission medium 13 has a flat surface, air may enter between the acoustic transmission medium 13 and the culture container 21. Therefore, if the surface of the acoustic transmission medium 13 in contact with the culture container 21 includes a convex curved surface, air is less likely to enter between the culture container 21 and the acoustic transmission medium 13.

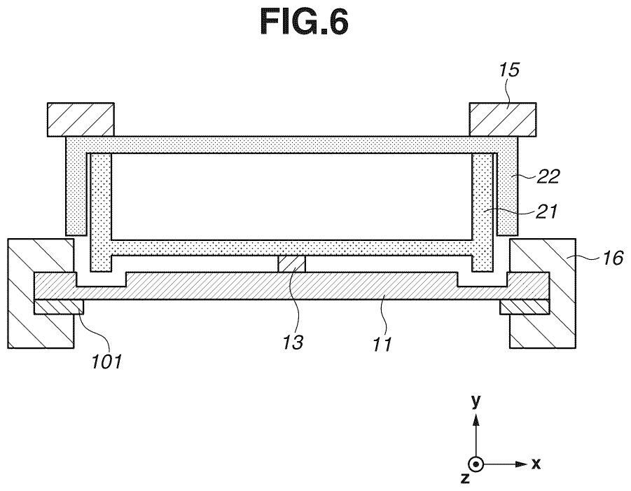

FIG. 6 is a schematic lateral view illustrating a center cross-section of the cell detachment apparatus 10 according to an exemplary embodiment of the present invention. A cover 22 is put on the culture container 21 placed on the cell detachment apparatus 10, and a ring shape weight 15 is placed on the cover 22. The vibrating plate 11 and the vibrator 101 are fixed by a ring shape fixing member 16 in a pinching manner. The weight 15 brings the culture container 21 and the acoustic transmission medium 13 into pressure contact with each other, and no air enters between the culture container 21 and the acoustic transmission medium 13, allowing the vibration of the vibrator 101 to be easily transmitted to the culture container 21.

### (Material of Acoustic Transmission Medium)

Preferably, the acoustic transmission medium 13 is made of a material that allows the vibration of the vibrator 101 to be easily transmitted to the culture container 21. In addition, the material also prevents air from entering the gap between the acoustic transmission medium 13 and the culture container 21, providing a high tacking property. Preferably, the acoustic transmission medium 13 contains a high-polymer material. For example, at least one material selected from a group including rubber, gel, and viscoelastic material can be used as the material of the acoustic transmission medium 13. Of these, rubber is preferable because of high vibration transmissibility and low price. Further, silicone rubber is preferable because of low attenuation in the drive frequency band, a high efficiency of vibration transmission, and the property to reduce heat development of the acoustic transmission medium 13 itself.

### (Method for Checking Positions of Antinodes and Nodes of Vibration Excited on Culture Container)

The following describes a method for checking the positions of the antinodes 17 or nodes 18 of the bending vibration having an arbitrary order excited on the culture container 21. In the state where the culture container 21 is empty, for example, in the configuration in FIGS. 1A, 1B, and 1C, a low-voltage input signal (e.g., an alternating current (AC) voltage of 1 to 10V) is input to the driving electrode 110 of the vibrator 101. Then, while changing the frequency of the input signal by using a laser doppler vibrometer, the vibration amplitudes at a plurality of points on the surface of the bottom surface 24 of the culture container 21 are measured for each frequency. More specifically, the vibration amplitudes of the bottom surface 24 can be measured, for example, by scanning the surface of the bottom surface 24 of the culture container 21 at 1-mm intervals. Of measurement points on the bottom surface 24, a portion having a minimal vibration amplitude is a node the position of which is a node 18, and a portion having a maximal vibration amplitude is an antinode the position of which is an antinode 17.

If the reflection of the laser from the surface of the culture container 21 is weak, and the measurement with the laser doppler vibrometer is difficult, the reflection of the laser can be strengthened by sticking a retroreflective sheet or depositing a metal such as platinum and gold on the surface subjected to the vibration amplitude measurement. However, performing vapor deposition decreases the survival rate of cells cultured in the culture container 21. In this case, a culture container 21 for the vibration amplitude measurement is prepared in addition to the culture container 21 for actual cell culturing, or the measurement can be performed after detaching the cells in the culture container 21.

### (Maintaining Positional Relation During Drive)

The configurations in FIGS. 1A, 1B, and 1C and FIGS. 3A and 3B enable transmitting the vibration of the vibrator 101 to the antinodes 17 of the bending vibration on the culture container 21 to detach the cells in the culture container 21. However, if the arrangements of the vibrator 101, the vibrating plate 11, and the acoustic transmission medium 13 change during driving, the efficiency of vibration transmission may be possibly reduced. Accordingly, when driving the cell detachment apparatus 10 according to an exemplary embodiment of the present invention, preferably, the three different members (the vibrator 101, the vibrating plate 11, and the acoustic transmission medium 13) are fixed to each other. These members may be fixed by using the fixing member 16 in FIG. 6 or bonded to each other by using an adhesive. In addition, each member may be fixed to other parts by using an adhesive. Preferably, felt, sponge, or rubber may be used as the fixing member 16 not to disturb the vibrations of the vibrator 101 and the vibrating plate 11.

### (Culture Container)

The material of the culture container 21 is not particularly limited. Examples of the materials of the culture container 21 include at least one material selected from a group including polyethylene, polypropylene, polycarbonate, polystyrene, polyvinyl chloride, nylon, polyurethane, polyurea, polylactic acid, polyglycolic acid, polyvinyl alcohol, polyvinyl acetate, poly(meta) acrylic acid, poly(meta) acrylic acid derivative, polyacrylonitrile, poly(meta) acrylamide, poly(meta) acrylamide derivative, polysulfone, cellulose, cellulose derivative, polysilicone, polymethylpentene, glass, and metals. Of these, polystyrene is preferable.

Types of the culture container 21 include dish, flask, and well plate. With the dish type in particular, products from Corning Incorporated, Thermo Fisher Scientific Inc., and AGC TECHNO GLASS CO., LTD. (IWAKI) of Japan are used as general-purpose products. The culture surface of the culture container 21 is the surface surrounded by the side wall. Cells adhere to the culture surface during cell culturing.

### (Cell Culture Conditions)

Cell culture conditions can be suitably selected according to the cells to be cultured. Generally, an appropriate culture medium is added in the culture container 21, and cells are seeded with a density of 1.0×10¹ to 5.0×10⁴ cells/cm² and cultured at 37° C. and with the CO₂ concentration of 5%. In this case, preferably, cells are cultured until the cell occupation area rate in the culture container 21 reaches about 70 to 80%, i.e., what is called the sub confluent state is attained. Cells may be cultured until they cover the entire culture surface (confluent state) to form a cell sheet.

### (Detachment Test)

The cell detachment apparatus 10 can be applied to various types of cells. Examples of cells include at least one cell type selected from a group including Chinese hamster ovary (CHO) cells, mouse connective tissue L929 cells, mouse skeletal muscle myoblast (C2C12 cells), human fetal lung normal diploid fibroblast cells (TIG-3 cells), human fetus kidney cells (HEK293 cells), human alveolar base epithelium glandular cancer A549 cells, human cervical cancer HeLa cells, epithelial cells, endothelial cells, skeletal muscle cells, smooth muscle cells, cardiac muscle cells, neuronal cells, glia cells, fibroblast cells, liver parenchyma cells, liver non-parenchyma cells, fat cells, derivative multipotent stem (iPS) cells, embryo-stem(ES) cells, embryo-generative (EG) cells, embryo-cancer (EC) cells, mesenchymal stem cells, liver stem cells, pancreas stem cells, skin stem cells, muscle stem cells, generative stem cells, precursor cells of each tissue, and cells differentiated and derived from the above-described cells.

The culture medium of the cultured cells needs to be replaced with a detachment solution in the cell detachment process. The detachment solution usually contains proteolytic enzymes. Examples of proteolytic enzymes include at least one enzyme selected from a group including trypsin, accutase, collagenase, natural protease, chymotrypsin, elastase, papain, pronase, and recombinants thereof. However, there is a concern about the possibility that proteolytic enzymes decompose the cellular membrane protein. The cell detachment technique according to the present invention exhibits a similar effect without using proteolytic enzymes or by using a very small amount of a proteolytic enzyme, providing an effect of reducing the decomposition of the cellular membrane protein. The detachment solution may contain culture medium components to prevent the reduction of the cellular membrane protein.

The culture container 21 containing the detachment solution replaced with the culture medium is set on the cell detachment apparatus 10, and the cell detachment apparatus 10 is driven while being maintained, for example, at a constant temperature (e.g., 37° C.) in an incubator. The cell detachment apparatus 10 is driven for a time duration sufficient for cell detachment. After the drive, the detached cells are collected with a dropping pipet, and cells adhering to the culture container 21 are collected through the pipetting method. The cell detachment efficiency refers to the ratio of the number of cells detached by the vibration of the cell detachment apparatus 10 to the sum total of the number of cells detached by the vibration and the number of cells detached by the pipetting method. The survival rate refers to the ratio of the number of living cells to the number of collected cells.

The remaining cells on the bottom surface of the culture container 21 after the cell detachment can be observed by using diffuse light produced with back light.

### (Other Configuration 1)

The cell detachment apparatus according to an exemplary embodiment of the present invention may include a plurality of acoustic transmission media as the acoustic transmission medium 13. The acoustic transmission medium 13 may have a non-discoid shape, e.g., a ring shape.

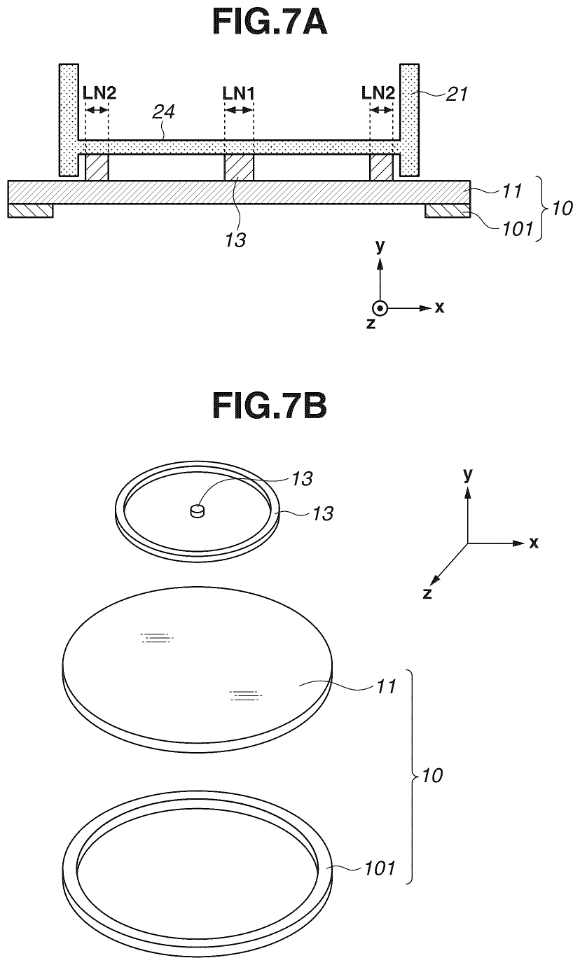

FIGS. 7A and 7B are schematic views illustrating another example of the cell detachment apparatus 10 according to an exemplary embodiment of the present invention. FIG. 7A is a schematic lateral view illustrating a center cross-section of the cell detachment apparatus 10 according to an exemplary embodiment of the present invention. FIG. 7B is an exploded perspective view illustrating a cell detachment apparatus 10 in FIG. 7A. FIG. 7B illustrates acoustic transmission media 13, a vibrating plate 11, and a vibrator 101 separated in the center axis direction (the direction in which these members are stacked) of the ring type vibrator 101.

FIGS. 7A and 7B illustrate an exemplary embodiment where the acoustic transmission medium 13 and the vibrating plate 11 have different shapes from those illustrated in FIGS. 1A, 1B, and 1C. The thickness of the acoustic transmission media 13 is larger than the thickness of the acoustic transmission medium 13 in FIGS. 1A, 1B, and 1C. Since the projecting portion of the culture container 21 will not come into contact with the vibrating plate 11, the discoid vibrating plate 11 is not provided with a groove. In addition, as the acoustic transmission media 13, a discoid acoustic transmission medium and a ring shape acoustic transmission medium 13 are disposed to come into contact with the culture container 21 at the antinodes 17 of the bending vibration. This form also provides the effect of the present invention.

Preferably, LN2/LM is 1.0 or larger and 1.6 or less where LN2 denotes the width of the region where the ring shape acoustic transmission medium 13 and the culture container 21 come into contact with each other, and LM denotes the distance from the first antinode of the bending vibration to the first node of the bending vibration closest to the first antinode. The width of the region where the ring shape acoustic transmission medium 13 and the culture container 21 come into contact with each other refers to the distance between the inner and the outer circumferences of the region of the ring shape acoustic transmission medium 13 in the radial direction (the direction perpendicular to the direction in which these members are stacked). If LN2/LM is 1.0 or larger, the region subjected to the vibration transmission is enlarged, allowing the vibration of the vibrator 101 to be efficiently transmitted to the culture container 21. If LN2/LM is 1.6 or less, air hardly enters between the culture container 21 and the acoustic transmission media 13.

### (Other Configuration 2)

With the cell detachment apparatus according to an exemplary embodiment of the present invention, the acoustic transmission media 13 need to come into contact with the culture container 21 at at least a part of the antinodes 17 of the bending vibration but do not need to come into contact with the center of the culture container 21.

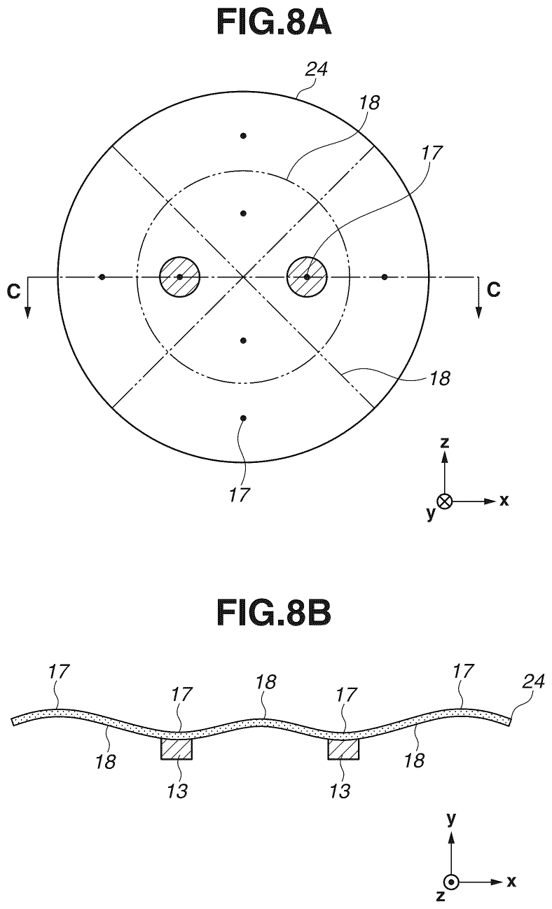

FIGS. 8A and 8B schematically illustrate a deformation state of other natural vibration mode in which the acoustic transmission media 13 and the bottom surface 24 of the culture container 21 enter a bending vibration state. FIG. 8A is a plan view illustrating the bottom surface 24 of the culture container 21 viewed from below the culture container 21. FIG. 8B is a lateral view illustrating a center cross-section (taken along the dot-dash line C-C of FIG. 8A) of the bottom surface 24 of the culture container 21 in the deformation state. However, referring to FIG. 8B, the deformation amount is illustrated with the increased deformation rate. Referring to 8A, the black dots denote antinodes 17, and the chain double-dashed lines denote nodes 18. Referring to FIGS. 8A and 8B, if the acoustic transmission media 13 are disposed at the positions corresponding to the antinodes 17 of the bending vibration on the bottom surface 24 of the culture container 21, but not disposed at the nodes 18, the acoustic transmission media 13 and the culture container 21 come into contact with each other at the antinodes 17 of the bending vibration, but do not come into contact at the nodes 18 of the bending vibration.

### (Other Configuration 3)

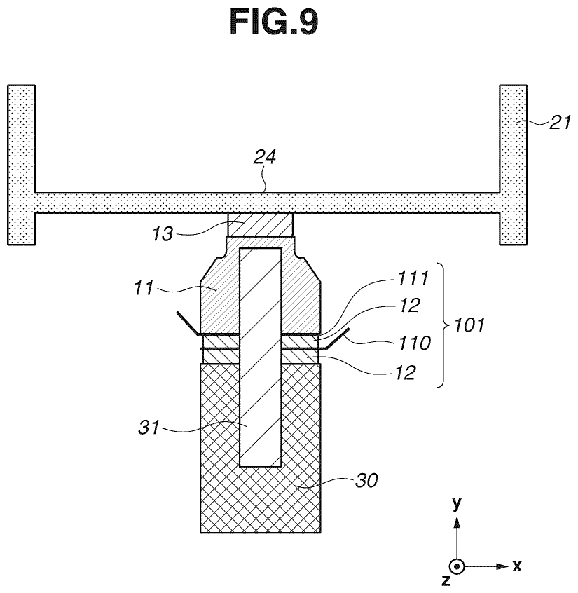

FIG. 9 is a schematic view illustrating the cell detachment apparatus 10 according to an exemplary embodiment of the present invention. A vibrator 101 is of the bolt-clamped Langevin type including ring shape piezoelectric materials 12, a driving electrode 110, and a common electrode 111. A metal block 30 and a vibrating plate 11 are connected by a bolt 31 at the center. When an alternating voltage is generated between the driving electrode 110 and the common electrode 111, the piezoelectric materials 12 expand and contract to cause the vibrating plate 11 to vibrate in the direction of a culture container 21. Thus, the vibrations of the vibrator 101 and the vibrating plate 11 are transmitted to antinodes 17 of the bending vibration excited on the bottom surface 24 of the culture container 21 via the acoustic transmission medium 13. In this case, the bending vibration on the bottom surface 24 of the culture container 21 is strongly excited, and the vibration amplitude of the bending vibration increases to detach the cells in the culture container 21.

### EXEMPLARY EMBODIMENTS

A first exemplary embodiment will be described below.

The cell detachment apparatus 10 in FIGS. 1A, 1B, and 1C is used for the first exemplary embodiment. A Φ60-mm dish from Thermo Fisher Scientific Inc. was used as the culture container 21. As the vibrating plate 11 of the cell detachment apparatus 10, a discoid glass plate with a diameter of 70 mm and a thickness of 4 mm was used. The glass plate is provided with a groove 14 so that the projecting portion 23 does not come into contact with the vibrating plate 11. The dimensions of the groove 14 were 50 mm in inner diameter, 5 mm in width, and 1 mm in depth. The piezoelectric material 12 had a material composition of PZT (C213 from Fuji Ceramics Co., Ltd.), an outer diameter of 70 mm (same as the outer diameter of the vibrating plate 11), an inner diameter of 57 mm, and a thickness of 2 mm. Silver was used as the driving electrode 110 and the common electrode 111. Then, the vibrator 101 and the vibrating plate 11 were bonded with an epoxy adhesive, and the discoid acoustic transmission medium 13 with a thickness of 0.8 mm and a diameter of 5 mm was disposed at the central portion on the vibrating plate 11. The material of the acoustic transmission medium 13 is silicone rubber having a hardness of 50 degrees.

Then, the cell detachment apparatus 10 was assembled as illustrated in FIG. 6, a Φ60-mm dish (Corning Incorporated) as the culture container 21 with cultured cells was placed on silicone rubber set on the vibrating plate 11, and a ring shape weight 15 of 300 g was placed on the dish. At this time, no air exists in the gap between the acoustic transmission medium 13 and the culture container 21, and a visual check was performed to confirm that the entire surface of the acoustic transmission medium 13 is in close contact with the culture container 21.

The vibrator 101 was swept with a voltage of 50V and at a frequency from 22 to 37 kHz, and the bottom surface 24 of the culture container 21 at each frequency was measured by using a laser doppler vibrometer (AT7200 from Graphtec Corporation). As a result, a standing wave of the bending vibration in FIG. 3A was produced at 24.3 kHz, and a standing wave of the bending vibration in FIG. 5A was produced at 35.1 kHz. However, in the vibration measurement, a culture container 21 with platinum deposited on the measurement surface was separately prepared.

Then, Chinese hamster ovary (CHO) cells were seeded with a density of 10,000 cells/cm² in the Φ60-mm dish (Thermo Fisher Scientific Inc.) prepared separately from the dish for the laser measurement, and then cultured at 37° C. and with the CO₂ concentration of 5%. As the culture medium, Ham's F12 (Thermo Fisher Scientific Inc.) with additives of Fetal Bovine Serum (Sigma-Aldrich) of 10% and penicillin-streptomycin (10,000 U/ml, Thermo Fisher Scientific Inc.) of 1% was used. After the cells were cultured for 48 hours, the cell state was observed by using a phase contrast microscope, and the cell adhesion and proliferation were confirmed. The cell occupation area rate of the dish was about 80%.

The culture medium in the dish was removed, and the dish was cleaned by using a phosphate buffered saline (PBS(−) from Thermo Fisher Scientific Inc.). Then, a phosphate buffered saline (PBS(−) from Thermo Fisher Scientific Inc.) as a detachment solution was added to the dish, and the dish was immersed for three minutes.

Then, the culture container 21 was placed on the cell detachment apparatus 10, and cells were detached by applying a frequency sweep vibration (at a frequency from 22 to 37 kHz, at frequency sweep intervals of 1 second, and with a voltage of 100V) for one minute and 30 seconds. In this case, when the temperature of the detachment solution was measured with a thermocouple, the temperature rise before cell detachment was 1° C. or less.

After the detached cells were collected, the survival rate was calculated with the life and death determination method based on the cell number measurement and the trypan blue dyeing by using an erythrocytometer. Firstly, a suspension of detached cells and a 0.4% trypan blue solution were mixed with a ratio of 1:1, and dead cells included in the suspension were dyed. Subsequently, the suspension was injected in an erythrocytometer (DHC-N01 from NanoEntek), and the numbers of living and dead cells on the grid of the erythrocytometer were counted by microscopy. The ratio of the number of counted living cells to the total number of cells was calculated to obtain the survival rate.

For the dish after ultrasonic wave detaching, all of cells that have not been detached with an ultrasonic wave were detached by using a cell scraper. The suspension of detached cells was injected to the erythrocytometer (DHC-N01 from NanoEntek) and the number of detached cells was measured by microscopy. The total number of cells detached by an ultrasonic wave and the number of cells detached by a cell scraper were added to obtain the total number of detached cells. The cell detachment efficiency defined as the ratio of the number of cells detached by an ultrasonic wave to the total number of detached cells was calculated. As a result, the survival rate was 95%, and the cell detachment efficiency was 91%. When both the survival rate and the cell detachment efficiency were 90% or higher, the result was determined to be favorable.

Exemplary embodiments of the present invention can be implemented by a method executed by a computer of a system or apparatus when the computer of the system or apparatus (e.g., application specific integrated circuit (ASIC)) for reading and executing computer-executable instructions (e.g., one or more programs) recorded on a storage medium executes one or a plurality of functions of the above-described exemplary embodiments, and/or when the computer of the system or apparatus including one or a plurality of circuits for executing one or the plurality of functions of the above-described exemplary embodiments reads computer-executable instructions from the storage medium and executes them, for example, to execute one or the plurality of the above-described exemplary embodiments, and/or when one or the plurality of circuits is controlled to execute one or the plurality of functions of the above-described exemplary embodiments. The computer can include one or more processors (e.g., central processing unit (CPU) and micro processing unit (MPU)) and, to read and execute computer-executable instructions, can include a network of different computers or different processors. Computer-executable instructions may be supplied from the network or storage medium to the computer. Examples of storage media include at least one of a hard disk, random access memory (RAM), read only memory (ROM), storage device of a distributed computing system, optical disk (including compact disc (CD)), Digital Versatile Disc (DVD), and Blu-ray Disc (BD)), flash memory device, and memory card.

The disclosure of the present embodiment includes the following configurations and methods.

### (Configuration 1)

A cell detachment apparatus that detaches cells placed on a culture surface of a culture container for cells by applying vibration to the cells. The cell detachment apparatus includes a vibrator and a vibrating plate. The vibrator, the vibrating plate, an acoustic transmission medium, and the culture container are stacked in this order. A bending vibration is excited on the culture container when a vibration of the vibrating plate is transmitted to the culture container via the acoustic transmission medium. The acoustic transmission medium and the culture container come into contact with each other at a region including an antinode of the bending vibration and do not come into contact at a node of the bending vibration.

### (Configuration 2)

The cell detachment apparatus according to configuration 1, wherein a bending vibration having a plurality of antinodes is excited on the culture container, and the acoustic transmission medium and the culture container come into contact with each other at a region including at least a part of the plurality of antinodes.

### (Configuration 3)

The cell detachment apparatus according to configuration 1 or 2, wherein the vibration is a vibration in an ultrasonic range.

### (Configuration 4)

The cell detachment apparatus according to any one of configurations 1 to 3, wherein the acoustic transmission medium contains a high-polymer material.

### (Configuration 5)

The cell detachment apparatus according to configuration 4, wherein the polymer material is silicone rubber.

### (Configuration 6)

The cell detachment apparatus according to any one of configurations 1 to 5, wherein the surface of the acoustic transmission medium in contact with the culture container includes a convex curved surface.

### (Configuration 7)

The cell detachment apparatus according to any one of configurations 1 to 6, in a plane perpendicular to a stacking direction, a cross-section of the acoustic transmission medium is circular.

### (Configuration 8)

The cell detachment apparatus according to the configuration 7, wherein LN1/LM is 1.0 or larger and 1.6 or less, where LM denotes a distance from a first antinode of the bending vibration to a first node of the bending vibration closest to the first antinode, and LN1 denotes the diameter of a region where the acoustic transmission medium and the culture container come into contact with each other.

### (Configuration 9)

The cell detachment apparatus according to any one of configurations 1 to 6, wherein the acoustic transmission medium has a ring shape.

### (Configuration 10)

The cell detachment apparatus according to the configuration 9, wherein LN/LM is 1.0 or larger and 1.6 or less, where LM denotes a distance from a first antinode of the bending vibration to a first node of the bending vibration closest to the first antinode, and LN denotes the width of the region where the acoustic transmission medium and the culture container come into contact with each other in a direction perpendicular to the stacking direction.

### (Configuration 11)

The cell detachment apparatus according to any one of configurations 1 to 10, wherein the vibrating plate transmits a vibration via the acoustic transmission medium to excite a bending vibration having a plurality of different natural vibration modes on the culture container.

### (Configuration 12)

The cell detachment apparatus according to configuration 11, wherein the vibrator is driven at the frequency at which each of the plurality of different natural vibration modes is excited on the culture container.

### (Configuration 13)

The cell detachment apparatus according to configuration 11, wherein the vibrator is driven by sweeping a frequency in a range including the frequency at which each of the plurality of different natural vibration modes is excited on the culture container.

### (Configuration 14)

The cell detachment apparatus according to any one of configurations 1 to 13, wherein the culture container has a projecting portion on the surface opposite to the culture surface, and the cell detachment apparatus is configured so that the acoustic transmission medium and the vibrating plate do not come into contact with the projecting portion.

### (Configuration 15)

The cell detachment apparatus according to configuration 14, wherein the cell detachment apparatus is configured so that at least either one of the acoustic transmission medium and the vibrating plate comes into contact with the projecting portion via a buffer material.

### (Configuration 16)

The cell detachment apparatus according to configuration 14, wherein the vibrating plate is provided with a groove in a region including the position on the vibrating plate where the position of the projecting portion is projected in the stacking direction.

### (Configuration 17)

The cell detachment apparatus according to any one of configurations 1 to 16, wherein the vibrating plate contains at least one material selected from a group including glass, metals, and quartz.

### (Configuration 18)

The cell detachment apparatus according to any one of configurations 1 to 17, further including a control unit configured to drive the vibrator to apply a vibration to the cells.

### (Method 1)

A cell detachment method for applying a vibration to cells disposed on a culture surface of a cell culture container to detach the cells. The method includes applying a vibration to the cells by using a cell detachment apparatus having a vibrator and a vibrating plate, an acoustic transmission medium, and the culture container in a state where the vibrator, the vibrating plate, the acoustic transmission medium, and the culture container are stacked in this order, and applying a vibration to the cells in a state where a bending vibration is excited on the culture container when a vibration of the vibrating plate is transmitted to the culture container via the acoustic transmission medium, and the acoustic transmission medium and the culture container come into contact with each other at a region including an antinode of the bending vibration but do not come into contact at a node of the bending vibration.

### (Method 2)

The cell detachment method according to method 1, further including applying a vibration to the cells in a state where the bending vibration having a plurality of antinodes is excited on the culture container, and the acoustic transmission medium and the culture container come into contact with each other at a region including at least a part of the plurality of antinodes.

### (Method 3)

The cell detachment method according to method 1 or 2, further including applying a vibration to the cells by using an acoustic transmission medium having a circular cross-section in a plane perpendicular to the stacking direction as the acoustic transmission medium, in a state where LN/LM is 1.0 or larger and 1.6 or less, where LM denotes a distance from a first antinode of the bending vibration to a first node of the bending vibration closest to the first antinode, and LN1 denotes the diameter of a region where the acoustic transmission medium and the culture container come into contact with each other.

The present invention is not limited to the above-described exemplary embodiments but can be modified and changed in diverse ways without departing from the spirit and scope thereof. Therefore, the following claims are appended to disclose the scope of the present invention.

The present invention makes it possible to provide a cell detachment apparatus for detaching cells with a high cell detachment efficiency.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A cell detachment apparatus that detaches cells placed on a culture surface of a culture container for cells by applying vibration to the cells, the cell detachment apparatus comprising:
a vibrator; and
a vibrating plate,
wherein the vibrator, the vibrating plate, an acoustic transmission medium, and the culture container are stacked in this order,
wherein a bending vibration is excited on the culture container when a vibration of the vibrating plate is transmitted to the culture container via the acoustic transmission medium, and
wherein the acoustic transmission medium and the culture container come into contact with each other at a region including an antinode of the bending vibration and do not come into contact at a node of the bending vibration.

2. The cell detachment apparatus according to claim 1,
wherein a bending vibration having a plurality of antinodes is excited on the culture container, and
wherein the acoustic transmission medium and the culture container come into contact with each other at a region including at least a part of the plurality of antinodes.

3. The cell detachment apparatus according to claim 1, wherein the vibration is a vibration in an ultrasonic range.

4. The cell detachment apparatus according to claim 1, wherein the acoustic transmission medium contains a high-polymer material.

5. The cell detachment apparatus according to claim 4, wherein the polymer material is silicone rubber.

6. The cell detachment apparatus according to claim 1, wherein a surface of the acoustic transmission medium in contact with the culture container includes a convex curved surface.

7. The cell detachment apparatus according to claim 1, wherein, in a plane perpendicular to a stacking direction, a cross-section of the acoustic transmission medium is circular.

8. The cell detachment apparatus according to the claim 7, wherein LN1/LM is 1.0 or larger and 1.6 or less, where LM denotes a distance from a first antinode of the bending vibration to a first node of the bending vibration closest to the first antinode, and LN1 denotes the diameter of a region where the acoustic transmission medium and the culture container come into contact with each other.

9. The cell detachment apparatus according to claim 1, wherein the acoustic transmission medium has a ring shape.

10. The cell detachment apparatus according to the claim 9, wherein LN/LM is 1.0 or larger and 1.6 or less, where LM denotes a distance from a first antinode of the bending vibration to a first node of the bending vibration closest to the first antinode, and LN denotes the width of the region where the acoustic transmission medium and the culture container come into contact with each other in a direction perpendicular to the stacking direction.

11. The cell detachment apparatus according to claim 1, wherein the vibrating plate transmits a vibration via the acoustic transmission medium to excite a bending vibration having a plurality of different natural vibration modes on the culture container.

12. The cell detachment apparatus according to claim 11, wherein the vibrator is driven at a frequency at which each of the plurality of different natural vibration modes is excited on the culture container.

13. The cell detachment apparatus according to claim 11, wherein the vibrator is driven by sweeping a frequency in a range including a frequency at which each of the plurality of different natural vibration modes is excited on the culture container.

14. The cell detachment apparatus according to claim 1,
wherein the culture container has a projecting portion on a surface opposite to the culture surface, and
wherein the cell detachment apparatus is configured so that the acoustic transmission medium and the vibrating plate do not come into contact with the projecting portion.

15. The cell detachment apparatus according to claim 14, wherein the cell detachment apparatus is configured so that at least either one of the acoustic transmission medium and the vibrating plate comes into contact with the projecting portion via a buffer material.

16. The cell detachment apparatus according to claim 14, wherein the vibrating plate is provided with a groove in a region including a position on the vibrating plate where a position of the projecting portion is projected in the stacking direction.

17. The cell detachment apparatus according to claim 1, wherein the vibrating plate contains at least one material selected from a group including glass, metals, and quartz.

18. The cell detachment apparatus according to claim 1, further comprising a control unit configured to drive the vibrator to apply a vibration to the cells.

19. A method for detaching cells placed on a culture surface of a culture container for cells by applying vibration to the cells, the method comprising:
applying a vibration to the cells by using a cell detachment apparatus having a vibrator and a vibrating plate, an acoustic transmission medium, and the culture container in a state where the vibrator, the vibrating plate, the acoustic transmission medium, and the culture container are stacked in this order; and
applying a vibration to the cells in a state where a bending vibration is excited on the culture container when a vibration of the vibrating plate is transmitted to the culture container via the acoustic transmission medium, and the acoustic transmission medium and the culture container come into contact with each other at a region including an antinode of the bending vibration but do not come into contact at a node of the bending vibration.

20. The cell detachment method according to claim 19, further comprising applying a vibration to the cells in a state where the bending vibration having a plurality of antinodes is excited on the culture container, and the acoustic transmission medium and the culture container come into contact with each other at a region including at least a part of the plurality of antinodes.

21. The cell detachment method according to claim 19, further comprising applying a vibration to the cells by using an acoustic transmission medium having a circular cross-section in a plane perpendicular to the stacking direction as the acoustic transmission medium, in a state where LN/LM is 1.0 or larger and 1.6 or less, where LM denotes a distance from a first antinode of the bending vibration to a first node of the bending vibration closest to the first antinode, and LN1 denotes the diameter of a region where the acoustic transmission medium and the culture container come into contact with each other.
